(19) United States
(12) Patent Application Publication
(10) Pub. No.: US 2015/0366690 A1
(43) Pub. Date: Dec. 24, 2015

(54) STENT DELIVERY SYSTEM WITH ANCHORING GUIDE WIRE AND METHOD FOR DEPLOYING A SELF-EXPANDING STENT

(71) Applicant: Abbott Cardiovascular Systems Inc., Santa Clara, CA (US)

(72) Inventor: Rommel C. Lumauig, San Jose, CA (US)

(21) Appl. No.: 14/312,055

(22) Filed: Jun. 23, 2014

(51) Int. Cl. A61F 2/966; A61F 2/958; A61F 2/82

(52) U.S. Cl. CPC A61F 2/966; A61F 2/82; A61F 2/958

(57) ABSTRACT

A stent delivery system includes a delivery catheter and an anchoring guide wire with an associated expandable member designed to expand within the patient's body vessel to anchor at least a distal portion of the delivery catheter within the body vessel. The anchoring of the distal portion of the delivery catheter helps to prevent a self-expanding stent, mounted on the delivery catheter, from jumping longitudinally in the body vessel as the outer restraining member of the catheter is being retracted since a portion of the self-expanding stent remains pressed against the wall of the body vessel allowing the stent to expand laterally with the vessel.

### BACKGROUND

This invention relates to the field of delivery systems for advancing intraluminal devices, such as stent delivery catheters, within body vessels in order to implant a medical device, such as an endoprostheses, more commonly referred to as an intravascular stent, into a patient's body vessel, such as a blood vessel or artery, to maintain the patency thereof. More particularly, the present invention is directed to a stent delivery system including a delivery catheter and anchoring feature that allows a self-expanding stent to expand laterally within a body lumen. The present invention also is directed to methods for laterally deploying a self-expanding stent within a body vessel.

Stents are generally tubular-shaped devices which function to hold open a segment of a blood vessel or other body lumen, such as a coronary or peripheral artery. They also are suitable for use to support and hold back a dissected arterial lining that can occlude the fluid passageway. At present, there are numerous commercial stents being marketed throughout the world. While some of these stents are flexible and have the appropriate radial rigidity needed to hold open a vessel or artery, there typically is a tradeoff between flexibility and radial strength.

Prior art stents typically fall into two general categories of construction. The first type of stent is expandable upon application of a controlled force, often through the inflation of the balloon portion of a dilatation catheter which, upon inflation of the balloon or other expansion means, expands the compressed stent to a larger diameter to be left in place within the artery at the target site. The second type of stent is a self-expanding stent formed from shape memory metals or super elastic nickel titanium (NiTi) alloys, which will automatically expand from a compressed state when the stent is advanced out of the distal end of the delivery catheter into the blood vessel. Such stents manufactured from expandable heat sensitive materials usually allow for phase transformations of the material to occur, resulting in the expansion and contraction of the stent.

Stents can be implanted in the coronary arteries along with peripheral arteries, such as the renal arteries, the carotid arteries and in long arterial segments in the leg, all of which are susceptible to arteriosclerosis. Generally, balloon-expandable stents have been implanted in the coronary arteries since the coronary arteries are generally not vulnerable to bending and compression forces that can distort the stent structure. Typically, balloon-expandable stents are made from a stainless steel, cobalt-chromium alloy, polymers, multi-layer materials or other similar biocompatible materials. Peripheral vessels, on the other hand, are usually more prone to natural bending and compressive forces which can easily bend and distort the implanted stent, causing it to fracture. For this reason, self-expanding stents are usually implanted in peripheral vessels since the self-expanding properties of the stent allows it to spring back to shape even after being subjected to bending or compressive forces.

Peripheral Artery Disease, or PAD, is characterized by fatty plaque build-up in the arteries of the legs, which results in poor blood flow and circulation. Patients with PAD may experience muscle pain during walking, have wounds and ulcers that are slow to heal or, in the most severe cases, require amputation of the legs. Possible treatments for PAD include lifestyle modification (including cessation of smoking), medicines, balloon dilatation, metal stent placement or bypass surgery.

One treatment of peripheral artery disease, for example, within the superficial femoral arteries, is the placement of a self-expanding stent utilizing a stent delivery system. Such a system may require the retraction of an outer sheath relative to an inner member over which a self-expanding stent is mounted. In a stent delivery system which deploys a self-expanding stent, the stent is usually collapsed or compressed to a smaller delivery diameter on an inner member of the delivery system and is covered by an outer restraining sheath or member which prevents the stent from self-expanding. The outer restraining sheath or member is then retracted from the self-expanding stent to allow the stent to expand to its larger deployed diameter. During deployment of the stent, however, a phenomenon known as “stent jumping” may occur due to difficulties in maintaining the inner member and stent completely stationary while retracting the outer restraining member. This stent jumping results in the self-expanding stent springing forward as the retraining sheath is being retracted from the collapsed stent which may result in inaccuracy of deployment of the self-expanding stent. In some cases, re-intervention may be required to ensure that the diseased arterial areas are properly scaffolded by the deployed stent.

Peripheral stents can be much longer than coronary stents since longer segments of the peripheral artery are usually required to be treated. The current trend for manufacturing peripheral stents is moving towards a longer stent, typically about 80-120 mm and longer, to treat long arterial segments in patients with critical limb ischaemia (CLI) in such arteries as, for example, the superficial femoral artery (SFA), along with arteries below the knee. Due to the lengths of these stents, it is important to ensure accurate placement in the target body vessel.

What has been needed and heretofore unavailable is a stent delivery system which has a high degree of flexibility so that it can be advanced through the often tortuous passageways of the patient's vasculature and can be radially expanded in a body segment that is susceptible to physiological deformations, and yet possesses the ability to accurately deploy the self-expanding stent at the target location within the patient by eliminating or curtailing the phenomenon known as stent jumping. The present invention satisfies these and other needs.

### SUMMARY OF THE INVENTION

The present invention is directed to a stent delivery system including a delivery catheter having an anchoring feature which allows the stent to expand laterally within a body vessel thus preventing longitudinal movement (stent jumping) from occurring. In one aspect of the invention, the delivery system includes a delivery catheter and an anchoring guide wire having an associated expandable member (the anchoring feature) designed to expand within the patient's body vessel in order to anchor at least a distal portion of the delivery catheter within the body vessel. The anchoring of the distal portion of the delivery catheter helps to prevent the self-expanding stent, which is mounted on the delivery catheter, from jumping distally as the outer restraining member of the catheter is being retracted to deploy the stent by allowing the stent to expand laterally within the body vessel. Thus, deployment of the self-expanding stent occurs in a more lateral fashion within the body vessel.

In one aspect, the delivery catheter includes an anchoring guide wire lumen which receives the anchoring guide wire and its associated expandable member. The distal end of the anchoring guide wire and the expandable member may extend out of a distal exit port of the anchoring guide wire lumen to allow the expandable member to be positioned relative to the outer surface of the inner member to allow the expandable member to press laterally against a distal portion of the inner member of the delivery catheter causing a portion of the distal portion to be pressed against the wall of the body vessel. In this manner, the distal portion of the inner member will be pressed between the expanding member and a portion of the wall of the body vessel thus preventing the distal portion of the inner member from moving (“anchoring the distal portion of the inner member”) when the outer retraining member is being retracted from the self-expanding stent. The anchoring of the inner member also places a portion of the self-expanding stent immediately adjacent the wall of the body vessel to help restrain the stent and prevent the stent from jumping longitudinally as the outer retraining member is being retracted. Accordingly, a stent coupled or mounted to the inner member of the delivery catheter actually deploys outwardly away (laterally) from the inner member as the outer restraining member is retracted, rather than “jumping” longitudinally within the body vessel. As a result, there is little chance of the self-expanding stent jumping distally when the restraining sheath is being retracted.

In one particular aspect, the anchoring guide wire lumen of the delivery catheter system may include a notch or exit port or opening to allow passage of the anchoring guide wire from the lumen to the outside of the inner member. For example, the inner member may include a lumen sized and configured to receive the anchoring guide wire and its associated expandable member, and the lumen may include an exit ramp that directs the anchoring guide wire and expandable member out of the inner member to laterally position the expandable member adjacent to the distal portion of the delivery catheter.

In another aspect of the present invention, the inner member of the delivery catheter may include a guide wire lumen for receiving a standard guide wire. the delivery system would include a standard guide wire which may be initially advanced through the body vessel to position the distal end of the standard guide wire into the target area. Thereafter, the stent delivery system may be advanced over the standard guide wire to the target site. The anchoring guide wire and its associated expandable member can then be deployed into the patient's body vessel to anchor the distal portion of the delivery catheter at the target site. The outer retraining member of the catheter can then be retracted to allow the self-expanding stent to expand with the body vessel.

In one particular aspect of the present invention, the anchoring guide wire includes a guide wire with the anchoring feature located near the distal end of the guide wire. The expandable member (“anchoring feature”) may be, for example, an inflatable balloon or an expandable cage. The anchoring feature may be deployed into contact with the vessel wall, and this contact may cause the anchoring feature to anchor both the anchoring guide wire and distal portion of the delivery catheter within the vessel.

In another aspect, once the anchoring guide wire is deployed to anchor the stent delivery catheter against the vessel wall, the outer restraining member may be retracted to allow the stent to deploy outwardly against the target lesion. Advantageously, since the inner member remains stationary during outer sheath retraction, any slack in the system or excessive longitudinal force applied to the stent by the outer sheath may be counteracted to prevent stent jumping.

In one aspect of the invention, a method for implanting a stent into a body vessel at a target location utilizes a stent delivery system which includes a stent delivery catheter carrying a self-expanding stent in a delivery configuration and a retractable outer restraining member which maintains the self-expanding stent in the delivery configuration. The method includes advancing the stent delivery catheter into the target location of the body vessel, maintaining at least a portion of the outer restraining member stent against a portion of the wall of the body vessel while the self-expanding stent remains in the delivery configuration and retracting the outer restraining member to allow a portion of the self-expanding stent to expand laterally within the body lumen to an expanded configuration.

In another aspect of the present invention, a method for implanting a stent into a body vessel at a target location utilizes a stent delivery system which includes a stent delivery catheter with an inner member having a distal portion and a stent mounting region for mounting a stent thereon, a stent mounted on the stent mounting region, the inner member having an anchoring guide wire lumen with an exit port defined therein, the system including an anchoring guide wire with an expandable member. The method includes advancing the stent delivery catheter to the target location in the body vessel, advancing the expandable member of the anchoring guide wire out of the exit port of the inner member, expanding the expandable member between the distal portion of the inner member and the wall of the body vessel to cause at least a portion of the distal portion of the inner member to contact the wall of the body vessel and implanting the stent within the body vessel. In another aspect, the delivery system includes a retractable outer member co-axially disposed over the inner member and the stent, the outer member being adapted for axial movement with respect to the inner member. This method further includes retracting the outer member after the expandable member has been expanded.

The method includes restraining a portion of the retractable outer restraining member between the wall of the body vessel and the expandable member after the expandable member is expanded. The method further includes retracting the outer restraining member after the expandable member has been expanded to uncover the stent. At least a portion of the self-expanding stent remains in contact with the stent mounting region and the wall of the body vessel after the outer member has been retracted. This causes the stent to be pinched between the expandable member and the wall as the outer restraining member is being retracted which helps to prevent the stent from jumping as the outer restraining member is retracted. As the outer restraining member is retracted, the self-expanding stent expands laterally in the body vessel away from the inner member of the delivery catheter.

In a further aspect, the method includes the use of a primary guide wire which is utilized to advance the catheter delivery system into the target region of the body vessel. In this aspect, the inner member of the delivery catheter includes a guide wire lumen which receives the primary guide wire. The primary guide wire is initially steered into the target region of the body vessel and then the delivery system is advanced over the guide wire into the target region.

Other features and advantages of the present invention will become more apparent from the following detailed description of the invention when taken in conjunction with the accompanying exemplary drawings.

### BRIEF DESCRIPTION OF THE DRAWINGS

FIG. 1 is an elevational view, partially in section, of one particular embodiment of a stent delivery system made in accordance with the present invention which includes a delivery catheter having a lumen for passing an anchoring guide wire into the target area of a patient's body vessel.

FIG. 2 is a cross sectional view of the stent delivery system of FIG. 1 taken along line 2-2.

FIG. 3 is an elevational view, partially in section, showing the distal end of the anchoring guide wire and the expandable member of the stent delivery system of FIG. 1 being advanced out of the anchoring guide wire lumen of the delivery catheter into the patient's body vessel.

FIG. 4 is an elevational view, partially in section, showing the expandable member of the stent delivery system of FIG. 1 being partially expanded within the patient's body vessel.

FIG. 5 is an elevational view, partially in section, showing the expandable member of the stent delivery system of FIG. 1 expanded within the patient's artery to anchor the distal portion of the stent delivery catheter within the patient's body vessel.

FIG. 6 is an elevational view, partially in section, showing the expandable member of the stent delivery system of FIG. 1 anchoring the distal portion of the stent delivery catheter within the patient's body vessel with the retractable outer member of the stent delivery system being partially retracted allowing the distal end of the self-expanding stent to begin lateral expansion within the patient's body vessel.

FIG. 7 is an elevational view, partially in section, showing the expandable member of the stent delivery system of FIG. 1 anchoring the distal portion of the stent delivery catheter within the patient's body vessel with the retractable outer member of the stent delivery system fully retracted from the stent to allow the stent to fully expand within the patient's artery.

FIG. 8 is an elevational view, partially in section, showing the expandable member of the stent delivery system as a fluid expandable balloon anchoring the distal portion of the stent delivery catheter within the patient's body vessel with the retractable outer member of the stent delivery system fully retracted from the stent to allow the stent to fully expand within the patient's body vessel.

### DETAILED DESCRIPTION OF THE PREFERRED EMBODIMENTS

The present invention provides a stent delivery system which includes a delivery catheter and an anchoring guide wire with an associated anchoring feature in the form of an expandable member designed to expand within the patient's body vessel so as to anchor at least a distal portion of the delivery catheter within a patient's body vessel. The anchoring of the distal portion of the delivery catheter helps to prevent the self-expanding stent, mounted on the delivery catheter, from jumping distally as the outer restraining member of the catheter is being retracted since the self-expanding stent will expand laterally within the body vessel. As a result, there is little chance of the self-expanding stent jumping distally (longitudinally within the body vessel) as the restraining sheath of the delivery system is being retracted. Accordingly, a stent mounted on the inner member of the delivery catheter deploys outwardly from the inner member as the outer restraining member is retracted, rather than “jumping” longitudinally within the vessel

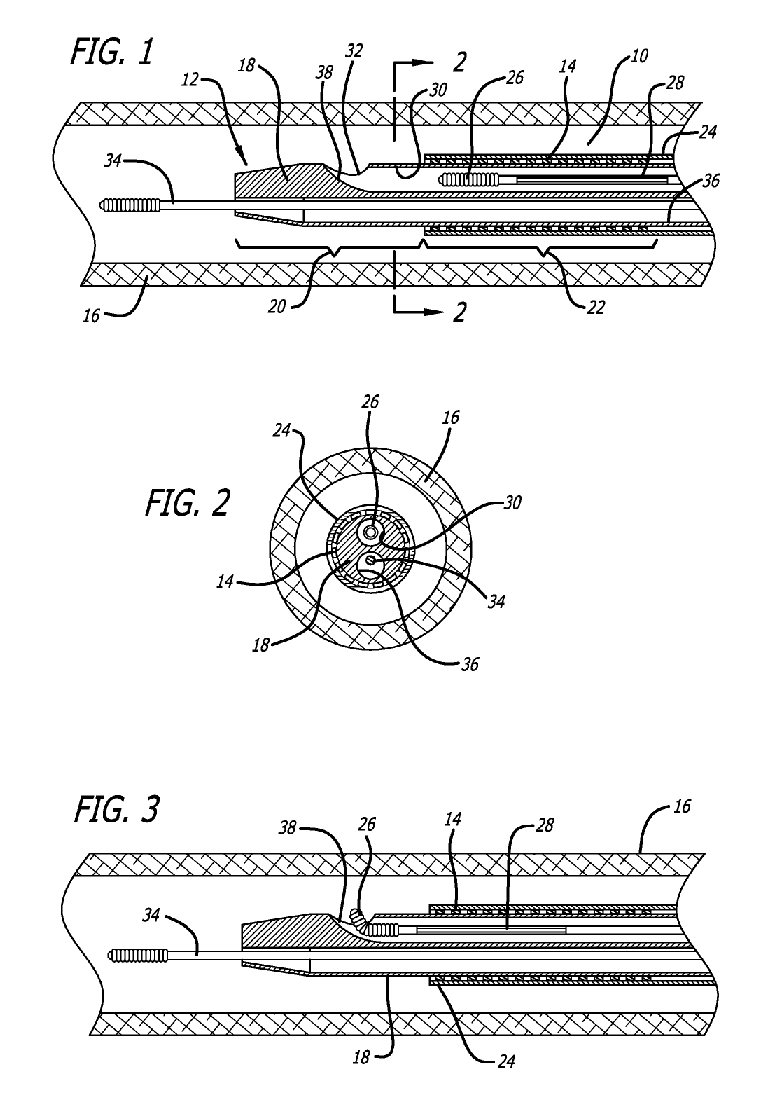

Turning to the drawings, FIGS. 1 and 2 depict a stent delivery system 10 made in accordance with the present invention. The stent delivery system 10 includes a stent delivery catheter 12 upon which a self-expanding stent 14 is mounted and is to be implanted at a target location within a patient's body vessel 16. The delivery catheter 12 includes an inner member 18 which includes a distal portion 20 and a stent mounting region 22 upon which the stent 14 is mounted. The delivery catheter 12 includes an outer restraining member 24 (also known as a “restraining sheath” in the art) which extends co-axially over the inner member 18 and the mounted stent 14. The outer restraining member 24 provides a means for restraining the self-expanding stent in its collapsed position or delivery configuration during delivery in the patient's body vessel. The outer restraining member 24 is retractable to allow the self-expanding stent 14 to gradually deploy as the outer restraining member 24 is being retracted. Both the inner member 18 and the outer retraining member 24 include proximal portions (not shown) which extend outside of the patient during usage. The proximal portions of the inner member 18 and outer restraining member 24 can be attached to a mechanism which allows the physician to retract the retraining member 24 from the self-expanding stent 14. Such mechanisms for retracting a restraining sheath relative to an inner member are well known in the art and exemplary devices are shown, for example, in U.S. Pat. Nos. 6,695,862 and 7,674,282, which are incorporated herein in their entirety. Similar structures for providing the physician with a means for retracting the outer restraining member can be incorporated with the disclosed delivery system.

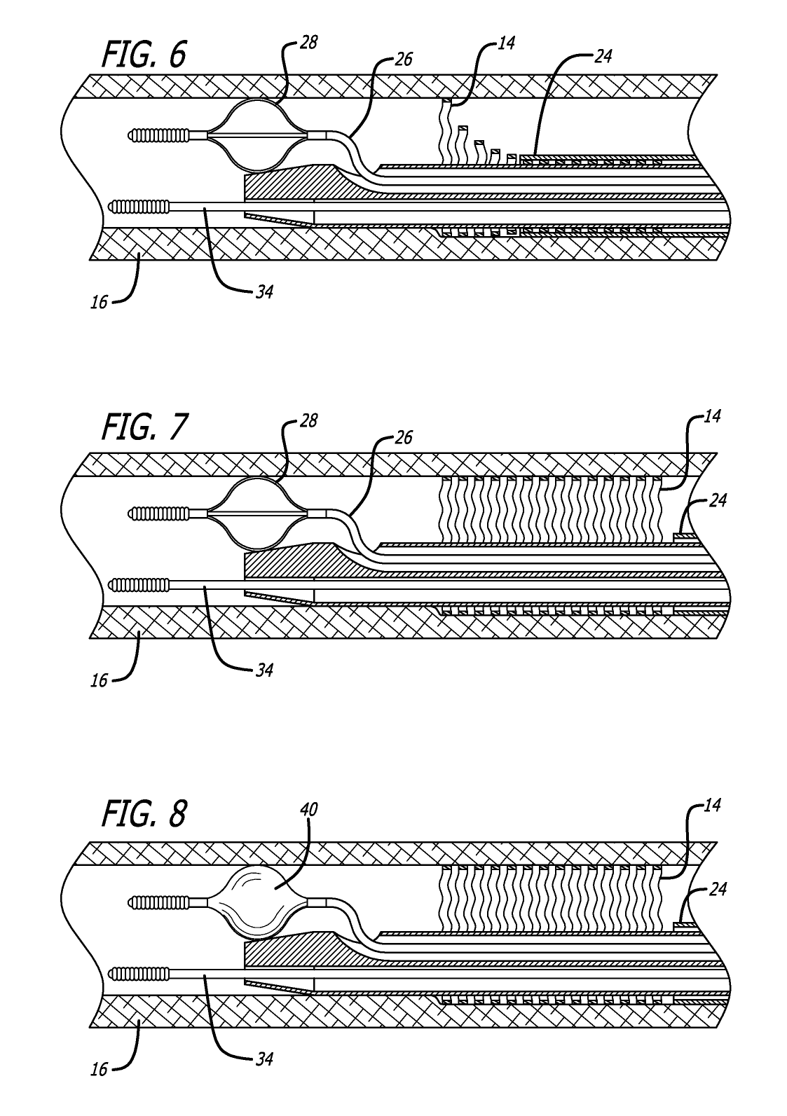

The stent delivery system 10 includes an anchoring guide wire 26 having an anchoring feature which is utilized to anchor the distal portion 20 of the inner member 18 within the body vessel 16. In the particular embodiment depicted in FIGS. 1-7, the anchoring feature comprises an expandable member 28 associated with the anchoring guide wire 26. This expandable member 28 (shown in its unexpanded condition in FIGS. 1 and 2) is designed to expand outside the distal portion 20 of the inner member 18 to anchor the delivery catheter in the body vessel 14 (as is shown in FIG. 7). This allows a portion of the self-expanding stent 14 to remain pressed against a portion of the wall of the body vessel 16 allowing the stent 14 to expand laterally as the outer restraining member 24 is retracted from the stent. Thus, there is little or no chance of the self-expanding stent jumping longitudinally within the body vessel as the restraining member 24 is being retracted from the stent 14.

The inner member 18 includes an anchoring guide wire lumen 30 which includes a notch or exit port 32 formed in the sidewall of the inner member 18. This lumen 30 is designed to receive both the anchoring guide wire 26 and its associated expandable member 28. This port 32 provides an opening through which the anchoring guide wire 26 and expandable member 28 can exit once the delivery catheter 12 is in place at the target region of the body vessel.

The stent delivery system 10 may further include a primary guide wire 34 which may be utilized to deliver the stent delivery system 10 into the target region of the body vessel. The inner member 18 may further include a primary guide wire lumen 36 which receives the primary guide wire 34. In usage, the primary guide wire 34 may be initially steered into the target region of the body vessel 16 allowing the delivery catheter 12 to be then advanced over the guide wire 34 into the target regions. Such techniques for advancing a stent delivery catheter over a guide wire are well known in the art.

Turning now to FIG. 3-7, various methods for anchoring the delivery catheter 12 in the patient's body vessel are disclosed. FIG. 3 shows the delivery system 10 after the delivery catheter 12 has been advanced into the target region of the body vessel 16. Initially, the primary guide wire 34 can be steered into the target region of the body vessel 16 utilizing techniques well known in the art. Once the distal end of the primary guide wire 34 is steered into place, the delivery catheter 12 can be advanced over the guide wire 34 into the target region of the body vessel 16. In an alternative procedure, the delivery catheter 12 could be advanced by itself into the target region of the body vessel in cases where the target region is in a relatively straight body vessel which does not need a primary guide wire 34 in order to advance the delivery catheter 12 into the target region. That being said, the primary guide wire 34 is particularly useful when the target region is located in the sometimes tortuous body vessels of the patient which require the delivery catheter 12 to be carefully steered into the target area.

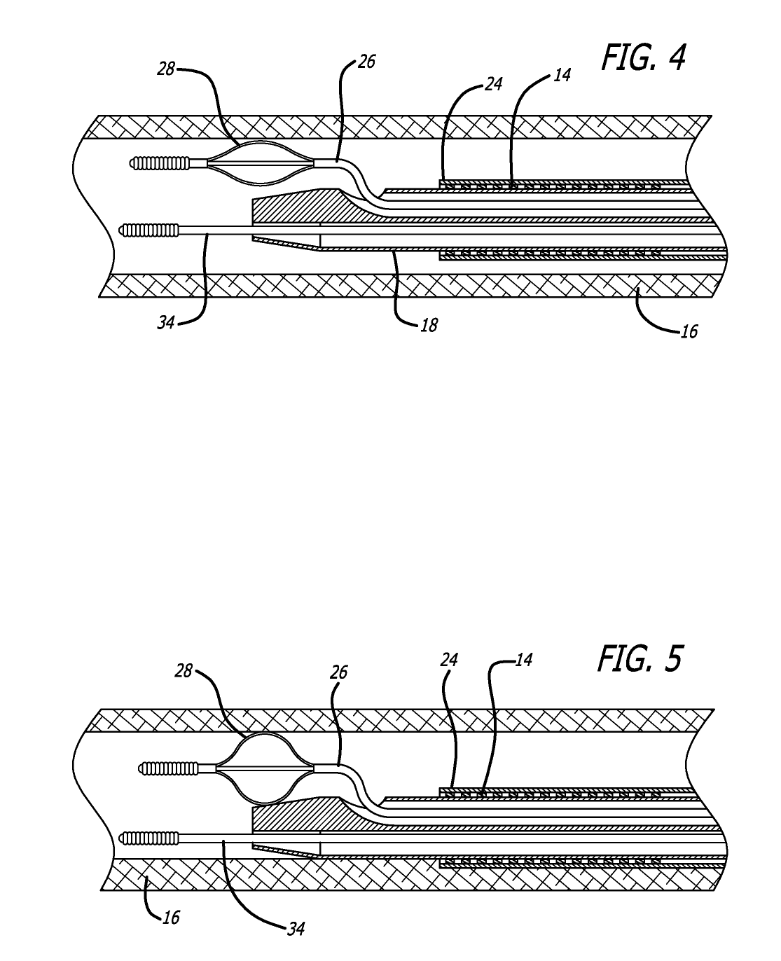

As can be seen in FIG. 3, the distal end of the anchoring guide wire 26 is shown being advanced through the exit port 32 of the anchoring guide wire lumen 30. The anchoring guide wire lumen 30 may include a ramp 38 designed to help direct the distal end of the anchoring guide wire 26 out of the lumen 30. Referring now to FIG. 4, the anchoring guide wire 26 and its associated expandable member 28 are shown advanced out of the lumen 30 a sufficient amount to allow the expandable member 28 to begin its expansion. As can be seen in FIGS. 4 and 5, the particular expandable member 28 is in the form of a cage-like device having expandable struts or arms which deploy from a collapsed position (see FIGS. 1 and 3) into an expanded position (see FIGS. 4-7). In FIG. 4, the expandable member 28 is not fully expanded as it is positioned adjacent to the distal portion 20 of the inner member 18.

Referring now to FIG. 5, the expandable member 28 is further expanded causing the inner member 18 to be pressed towards the wall of the body vessel 16 causing the delivery catheter 12 to be anchored in the body vessel. As can be seen in FIG. 5, the distal portion 20 of the inner member 18 is pressed against the wall of the body vessel 16 which prevents the inner member 18 from moving as the outer restraining member 24 is being retracted from the mounted stent 14. Also, at least a distal portion of the outer retraining member 24 is pressed up against the wall of the body vessel 16 as well. The significance of pressing a portion of the outer retraining member 24 can be seen in FIGS. 6 and 7 which shows the progressive, lateral movement of the self-expanding stent 14 as the outer restraining member 24 is being retracted.

As can be seen in FIG. 6, the outer restraining member 24 has been retracted from the self-expanding stent 14 allowing the distal portion of the stent 14 to begin its lateral expansion to the larger, deployed configuration. The significance of pressing the distal portion of the delivery catheter 12 against the wall of the body vessel 16 allows the stent 14 itself to be pressed against the wall of the body vessel 16 as well. Accordingly, there is little chance of the stent 14 jumping forward since at least a portion of the stent 14 is being pressed between the mounting region 22 of the inner member 18 and the wall of the body vessel 16. As can be seen in FIG. 6, as soon as the outer restraining member 24 is initially retracted, the distal end of the stent 14 begins to expand laterally within the body vessel 16. This allows the end of the stent 14 to be anchored within the body lumen 16 helping to prevent the stent 14 from jumping forward as the outer restraining member 24 is being retracted.

As the outer restraining member 24 continues to be retracted, as is shown in FIG. 7, the remainder of the self-expanding stent 14 expands laterally into the lumen of the body vessel while a portion of the stent 14 still remains pressed between the mounting region 22 and the wall of the body vessel 16. Again, the deployment of the stent 14 in this fashion helps to prevent the stent from jumping longitudinally from the inner member 18 as the outer restraining member 24 is being retracted.

FIG. 8 shows another form of an expandable member 40 which could be used with the anchoring guide wire 34. In this embodiment, the expandable member 40 is in the form of an inflatable balloon which can be inflated by inflation fluid to allow the balloon expand against the delivery catheter 12. In such an embodiment, the anchoring guide wire 26 would require an inflation lumen (not shown) for filling the balloon with an inflation fluid.

Alternatively, the expandable member 28 can be made from a self-expanding material as well which allows the expandable member 28 to begin expanding once the expandable member 28 is advanced out of the anchoring guide wire lumen 30. Accordingly, the anchoring guide wire lumen 30 acts a restraining sheath which maintains the expandable member 28 in its collapsed configuration until the expandable member 28 is advanced out of the exit port 32. Once the expandable member 28 moves out of the exit port 30, the self-expanding characteristics of the expandable member 28 causes the member 28 to begin expansion, causing the delivery catheter 12 to be pressed against the wall of the body vessel.

In the embodiment of the expandable member 28 shown in FIGS. 1-7, the expandable member 28 can be remotely expanded to the desired expanded configuration utilizing a number of expansion techniques. For example, the expandable member 28 could be formed from a tubing which extends outside of the patient. A length of wire could extend through the lumen of the tubing and be attached to the distal end of the expandable member 28. The physician can then pull on the wire causing inward axial forces to be applied on the ends of the expandable member causing the arms of the expandable member 28 to bulge outwardly into the expanded configuration as is shown in FIGS. 5-7. It will be appreciated that other forms of expandable members could be utilized with the anchoring guide wire without departing from the spirit and scope of the present invention.

The present stent delivery system can be utilized to deliver the stent 14 and implant it in a body vessel, such as a peripheral artery, a coronary artery or other vessel within the body. The delivery catheter 12 is configured to be advanced through the patient's vascular system by advancing the catheter 12 over the primary guide wire using well known methods associated with over-the-wire or rapid-exchange catheter systems. It should be appreciated that the delivery catheter 12 can include rapid-exchange structure which receives the various guide wires to eliminate the need for long tubular components. Such delivery catheters which utilize rapid-exchange technology with a retractable outer retraining sheaths are well known in the art.

The tubing of a self-expanding expandable member in accordance with the present invention may be made of suitable biocompatible material such as nickel-titanium (NiTi) alloys and other materials which possess self-expanding properties. The expandable member can be manufactured utilizing similar laser cutting techniques used to manufacture a stent. For example, one method of making the stent or expandable member is to cut a thin-walled tubular member, such as Nitinol tubing, and remove portions of the tubing in the desired pattern for the stent or expandable member, leaving relatively untouched the portions of the metallic tubing which are to form the stent or expandable member. The tubing can be cut in the desired pattern by means of a machine-controlled laser.

It should be appreciated the stent used with the delivery system of the present invention also can be a balloon expandable stent as well. In this case, the delivery catheter would be used with the anchoring guide wire to anchor the delivery system within the patient's body vessel. The delivery catheter would require a structure which allows an inflation fluid to inflate an expandable balloon used to expand the stent.

Although the present invention has been described in terms of certain preferred embodiments, other embodiments that are apparent to those of ordinary skill in the art are also within the scope of the invention. Accordingly, the scope of the invention is intended to be defined only by reference to the appended claims. While the dimensions, types of materials and coatings described herein are intended to define the parameters of the invention, they are by no means limiting and are exemplary embodiments.

What is claimed:

1. A stent delivery system for placing a stent within a body vessel, comprising:
a delivery catheter including an inner member having a distal portion and a stent mounting region for mounting a stent thereon, the inner member having an anchoring guide wire lumen with an exit port;
a stent mounted in the stent mounting region of the inner member; and
an anchoring guide wire having an expandable member which can pass through the exit port of the anchoring guide wire lumen, the expandable member being expandable outside of the inner member to press at least a portion of the distal portion of the inner member against the wall of the body vessel.

2. The stent delivery system of claim 1, further including a retractable outer restraining member overlying the inner member and adapted for axial movement with respect to the inner member, the outer member being adapted to overly the stent mounted in the stent mounting region.

3. The stent delivery system of claim 1, wherein the exit port of the anchoring guide wire lumen is located in the distal portion of the inner member.

4. The stent delivery system of claim 3, wherein the distal portion of the inner member has a side wall and the exit port is formed in the side wall of the inner member.

5. The stent delivery system of claim 4, wherein the exit port is located distal to the stent mounting region of the inner member.

6. The stent delivery system of claim 1, further including a ramp located in the anchoring guide wire lumen for directing the expandable member of the anchoring guide wire out of the exit port.

7. The stent delivery system of claim 1, wherein the expandable member of the anchoring guide wire is positionable with respect to the stent mounting region so that when the expandable member is expanded outside of the inner member, at least a portion of the stent mounting region is pressed against the wall of the body vessel.

8. The stent delivery system of claim 1, wherein expansion of the expandable member of the anchoring guide wire within the body vessel causes at least a portion of the distal portion of the inner member to contact the wall of the body vessel.

9. The stent delivery system of claim 8, wherein expansion of the expandable member of the anchoring guide wire within the body vessel causes at least a portion of the stent mounting region of the inner member to contact the wall of the body vessel.

10. The stent delivery system of claim 1, wherein the expandable member is fluid inflatable.

11. The stent delivery system of claim 1, wherein the expandable member is made from a self-expanding material.

12. The stent delivery system of claim 2, wherein the stent is a self-expanding stent mounted on the stent mounting region and wherein the outer restraining member is co-axially disposed over the self-expanding stent.

13. The stent delivery system of claim 12, wherein expansion of the expandable member of the anchoring guide wire within the body vessel causes at least a portion of the outer restraining member to contact the wall of the body vessel.

14. The stent delivery system of claim 1, wherein the inner member has a primary guide wire lumen and the system further includes a primary guide wire.

15. A method for implanting a stent into a body vessel at a target location utilizing a stent delivery system which includes a stent delivery catheter carrying a self-expanding stent in a delivery configuration and a retractable outer restraining member which maintains the self-expanding stent in the delivery configuration, the self-expanding stent having an expanded configuration, the method comprising:
advancing the stent delivery catheter into the target location of the body vessel;
maintaining at least a portion of the outer restraining member against a portion of the wall of the body vessel while the self-expanding stent remains in the delivery configuration; and
retracting the outer restraining member to allow the self-expanding stent to expand laterally within the body lumen to its expanded configuration.

16. The method of claim 15, wherein a portion of the self-expanding stent remains in contact with the wall of the body vessel as the outer restraining member is being retracted.

17. A method for implanting a stent into a body vessel at a target location utilizing a stent delivery system which includes a stent delivery catheter with an inner member having a distal portion and a stent mounting region for mounting a stent thereon, a self-expanding stent mounted in a delivery configuration on the stent mounting region, the self-expanding stent having an expanded configuration, the inner member having an anchoring guide wire lumen with an exit port defined therein, the system including an anchoring guide wire with an expandable member having a delivery configuration and expanded configuration, the method comprising:
advancing the stent delivery catheter into the target location of the body vessel;
advancing the expandable member of the anchoring guide wire out of the exit port of the anchoring guide wire lumen;
expanding the expandable member to its expanded configuration between the distal portion of the inner member and the wall of the body vessel to cause at least a portion of the distal portion of the inner member to contact the wall of the body vessel and to place at least a portion of the self-expanding stent adjacent to the wall of the body vessel while the self-expanding stent remains in its delivery configuration; and
allowing the self-expanding stent to expand laterally within the body vessel.

18. The method of claim 17, wherein the delivery system includes a retractable outer member co-axially disposed over the inner member and the self-expanding stent, the outer member being adapted for movement with respect to the inner member.

19. The method of claim 18, further including:
retracting the outer member after the expandable member has been expanded.

20. The method of claim 18, wherein a portion of the retractable outer member contacts the wall of the body vessel after the expandable member is expanded.

21. The method of claim 17, further including:
placing the expandable member back to its delivery configuration after the self-expanding stent has been implanted in the body vessel.

22. The method of claim 21, wherein at least a portion of the self-expanding stent remains in contact with the stent mounting region and the wall of the body vessel after the outer member has been retracted.

23. A method for implanting a stent into a body vessel at a target location utilizing a stent delivery system which includes a stent delivery catheter with an inner member having a distal portion, a proximal portion and a stent mounting region for mounting a stent thereon, a self-expanding stent mounted in a delivery configuration on the stent mounting region, the self-expanding stent having an expanded configuration, the inner member having a primary guide wire lumen and an anchoring guide wire lumen with an exit port defined therein, the system including a primary guide wire and an anchoring guide wire having an expandable member, the method comprising:
advancing the primary guide wire into the body vessel to the target location;
advancing the stent delivery catheter along the guide wire to the target location;
advancing the expandable member of the anchoring guide wire out of an exit port;
expanding the expandable member between the distal portion of the inner member and the wall of the body vessel to cause at least a portion of the distal portion of the inner member to contact the wall of the body vessel and to place at least a portion of the self-expanding stent adjacent to the wall of the body vessel while the self-expanding stent remains in its delivery configuration; and
allowing the self-expanding stent to expand laterally within the body vessel.

24. The method of claim 23, wherein the delivery system includes a retractable outer retraining member co-axially disposed over the inner member and the self-expanding stent, the outer member being adapted for movement with respect to the inner member.

25. The method of claim 24, further including:
retracting the outer retraining member after the expandable member has been expanded.

26. The method of claim 24, wherein a portion of the retractable outer member contacts the wall of the body vessel after the expandable member is expanded.

27. The method of claim 25, further including:
placing the expandable member back to its delivery configuration after the self-expanding stent has been implanted in the body vessel.

28. The method of claim 24, wherein at least a portion of the self-expanding stent remains in contact with the stent mounting region and the wall of the body vessel after the outer restraining member has been retracted.
